(19) United States
(12) Patent Application Publication
(10) Pub. No.: US 2010/0105705 A1
(43) Pub. Date: Apr. 29, 2010

(54) PURINYL DERIVATIVES AND THEIR USE AS POTASSIUM CHANNEL MODULATORS

(75) Inventors: Birgitte L. Eriksen, Farum (DK); Ulrik Svane, Soborg (DK); Charlotte Hougaard, Bagsvaerd (DK); Dan Peters, Malmo (SE); Tina Holm Johansen, Smorum (DK); Palle Christophersen, Ballerup (DK)

Correspondence Address: BIRCH STEWART KOLASCH & BIRCH, PO BOX 747, FALLS CHURCH, VA, 22040-0747, US

(73) Assignee: NeuroSearch A/S, Ballerup (DK)

(21) Appl. No.: 12/593,269

(22) PCT Filed: Mar. 27, 2008
(86) PCT No.: PCT/EP08/53649

§ 371 Date: Jan. 13, 2010

Related U.S. Application Data
(60) Provisional application No. 60/908,603, filed on Mar. 28, 2007.

(30) Foreign Application Priority Data
Mar. 28, 2007 (DK) PA 2007 00482

(51) Int. Cl. A61K 31/52; C07D 473/00

(52) U.S. Cl. 514/263.22; 544/264; 514/263.2

(57) ABSTRACT

This invention relates to novel purinyl derivatives and their use as potassium channel modulating agents. Moreover the invention is directed to pharmaceutical compositions useful for the treatment or alleviation of diseases or disorders associated with the activity of potassium channels.

### TECHNICAL FIELD

This invention relates to novel purinyl derivatives and their use as potassium channel modulating agents. Moreover the invention is directed to pharmaceutical compositions useful for the treatment or alleviation of diseases or disorders associated with the activity of potassium channels.

### BACKGROUND ART

Ion channels are transmembrane proteins, which catalyse the transport of inorganic ions across cell membranes. The ion channels participate in processes as diverse as the generation and timing of action potentials, synaptic transmissions, secretion of hormones, contraction of muscles, etc.

All mammalian cells express potassium (K⁺) channels in their cell membranes, and the channels play a dominant role in the regulation of the membrane potential. In nerve and muscle cells they regulate the frequency and form of the action potential, the release of neurotransmitters, and the degree of broncho- and vasodilation.

From a molecular point of view, the K⁺ channels represent the largest and most diverse group of ion channels. For an overview they can be divided into five large subfamilies: Voltage-activated K⁺ channels (Kᵥ), long QT related K⁺ channels (KvLQT), inward rectifiers (K_{IR}), two-pore K⁺ channels (K_{TP}), and calcium-activated K⁺ channels (K_{ca}).

The latter group, the Ca²⁺-activated K⁺ channels, consists of three well-defined subtypes: SK channels, IK channels and BK channels. SK, IK and BK refer to the single-channel conductance (Small, Intermediate and Big conductance K channel). The SK, IK, and BK channels exhibit differences in e.g. voltage- and calcium-sensitivity, pharmacology, distribution and function.

SK channels are present in many central neurons and ganglia, where their primary function is to hyperpolarize nerve cells following one or several action potentials, in order to prevent long trains of epileptogenic activity to occur. The SK channels are also present in several peripheral cells including skeletal muscle, gland cells, liver cells, and T-lymphocytes. The significance of SK channels in normal skeletal muscle is not clear, but their number is significantly increased in denervated muscle, and the large number of SK channels in the muscle of patients with myotonic muscle dystrophia, suggest a role in the pathogenesis of the disease.

Studies indicate that K⁺ channels may be a therapeutic target in the treatment of a number of diseases including asthma, cystic fibrosis, chronic obstructive pulmonary disease and rhinorrhea, convulsions, vascular spasms, coronary artery spasms, renal disorders, polycystic kidney disease, bladder spasms, overactive bladder, urinary incontinence, bladder outflow obstruction, interstitiel cystitis, irritable bowel syndrome, gastrointestinal dysfunction, secretory diarrhoea, ischaemia, cerebral ischaemia, ischaemic heart disease, angina pectoris, coronary heart disease, traumatic brain injury, psychosis, anxiety, depression, dementia, memory and attention deficits, Alzheimer's disease, dysmenorrhea, narcolepsy, Reynaud's disease, intermittent claudication, Sjogren's syndrome, migraine, pain, arrhythmia, hypertension, absence seizures, myotonic muscle dystrophia, xerostomi, diabetes type II, hyperinsulinemia, premature labour, baldness, cancer and immune suppression.

### SUMMARY OF THE INVENTION

The present invention resides in the provision of novel chemical compounds capable of modulating SK channels, or subtypes of SK channels.

Accordingly, in its first aspect, the invention provides novel purinyl derivative of Formula Ia or Ib

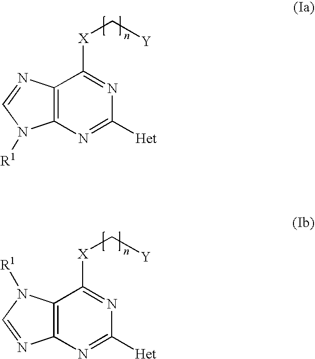

an isomer thereof or a mixture of its isomers, an N-oxide thereof, a prodrug thereof, or a pharmaceutically acceptable salt thereof, wherein
n is 0, 1, 2 or 3;
X represents O, S or NR′; wherein R′ represents hydrogen, alkyl, cycloalkyl, phenyl or benzyl;
Y represents alkyl, cycloalkyl or phenyl; which alkyl, cycloalkyl and phenyl are optionally substituted with one substituent selected from the group consisting of alkyl, cycloalkyl, halo, trifluoromethyl, trifluoromethoxy, hydroxy, alkoxy, cyano, nitro and amino;
R¹ represents hydrogen, alkyl or alkoxy-alkyl; and
Het represents a heterocyclic group selected from pyrazolyl, imidazolyl, indazolyl, benzimidazolyl, pyridinyl and cyclopentapyrazolyl, which pyrazolyl, imidazolyl, indazolyl, benzimidazolyl, pyridinyl and cyclopentapyrazolyl may optionally be substituted with one substituent selected from the group consisting of alkyl, hydroxy-alkyl, cyclo-alkyl, cycloalkyl-alkyl, alkenyl, alkynyl, halo, trifluoromethyl, trifluoromethoxy, hydroxy, alkoxy, alkoxy-carbonyl, carboxy, cyano, nitro, amino, amino-carbonyl, N,N-dialkyl-amino-carbonyl, phenyl and benzyl.

In another aspect, the invention provides pharmaceutical compositions comprising an effective amount of a compound of the invention.

In further aspects the invention relates to the use of a derivative of the invention for the manufacture of a medicament for the treatment or alleviation of diseases or disorders associated with the activity of potassium channels, and to method of treatment or alleviation of disorders or conditions responsive to modulation of potassium channels.

### DETAILED DISCLOSURE OF THE INVENTION

### Potassium Channel Modulating Agents

In its first aspect, the invention provides novel purinyl derivatives of Formula Ia or Ib

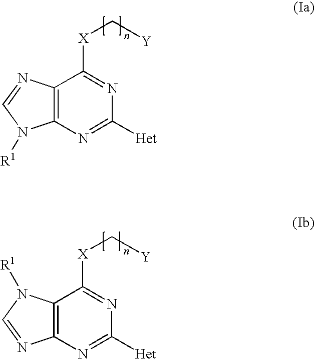

a stereoisomer thereof or a mixture of its stereoisomers, an N-oxide thereof, a prodrug thereof, or a pharmaceutically acceptable salt thereof, wherein
n is 0, 1, 2 or 3;
X represents O, S or NR′; wherein R′ represents hydrogen, alkyl, cycloalkyl, phenyl or benzyl;
Y represents alkyl, cycloalkyl or phenyl; which alkyl, cycloalkyl and phenyl are optionally substituted with one substituent selected from the group consisting of alkyl, cycloalkyl, halo, trifluoromethyl, trifluoromethoxy, hydroxy, alkoxy, cyano, nitro and amino;
R¹ represents hydrogen, alkyl or alkoxy-alkyl; and
Het represents a heterocyclic group selected from pyrazolyl, imidazolyl, indazolyl, benzimidazolyl, pyridinyl and cyclopentapyrazolyl, which pyrazolyl, imidazolyl, indazolyl, benzimidazolyl, pyridinyl and cyclopentapyrazolyl may optionally be substituted with one substituent selected from the group consisting of alkyl, hydroxy-alkyl, cyclo-alkyl, cycloalkyl-alkyl, alkenyl, alkynyl, halo, trifluoromethyl, trifluoromethoxy, hydroxy, alkoxy, alkoxy-carbonyl, carboxy, cyano, nitro, amino, amino-carbonyl, N,N-dialkyl-amino-carbonyl, phenyl and benzyl.

In another embodiment the derivative of the invention is a purinyl-pyrazole derivative of Formula IIa or IIb

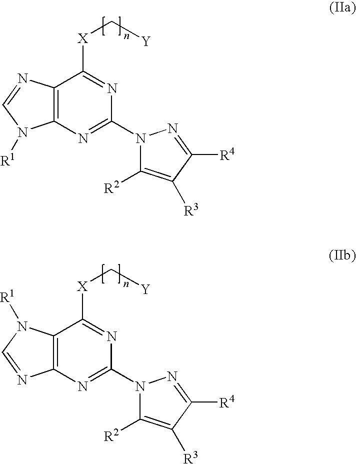

a stereoisomer thereof or a mixture of its stereoisomers, an N-oxide thereof, a prodrug thereof, or a pharmaceutically acceptable salt thereof, wherein
n, X, Y and R¹ are as defined above; and
R², R³ and R⁴ represent hydrogen; or
two of R², R³ and R⁴ represent hydrogen; and the remaining one of R², R³ and R⁴ represents alkyl, hydroxy-alkyl, cycloalkyl, cycloalkyl-alkyl, alkenyl, alkynyl, halo, trifluoromethyl, trifluoromethoxy, hydroxy, alkoxy, alkoxy-carbonyl, carboxy, cyano, nitro, amino, amino-carbonyl, N,N-dialkyl-amino-carbonyl, phenyl or benzyl.

In another embodiment the derivative of the invention is a purinyl-indazolyl derivative of Formula IIIa or IIIb

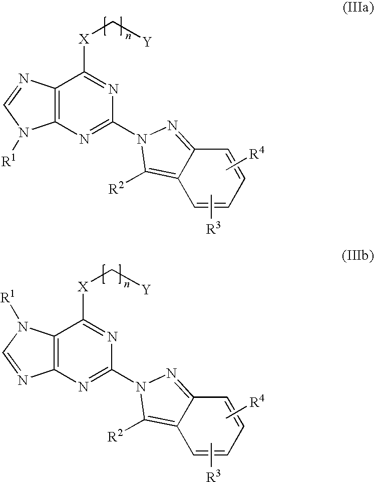

a stereoisomer thereof or a mixture of its stereoisomers, an N-oxide thereof, a prodrug thereof, or a pharmaceutically acceptable salt thereof, wherein n, X, Y, R¹, R², R³ and R⁴ are as defined above.

In another embodiment the derivative of the invention is a purinyl-indazolyl derivative of Formula IVa or IVb

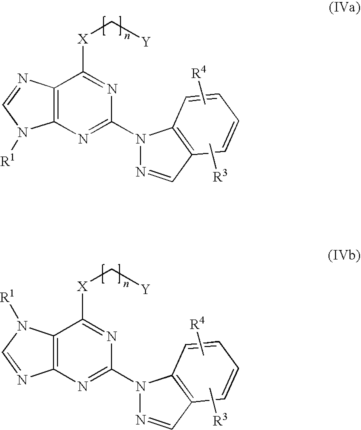

a isomer thereof or a mixture of its isomers, an N-oxide thereof, a prodrug thereof, or a pharmaceutically acceptable salt thereof, wherein n, X, Y, R¹, R², R³ and R⁴ are as defined above.

In another embodiment the derivative of the invention is a purinyl-benzimidazolyl derivative of Formula Va or Vb

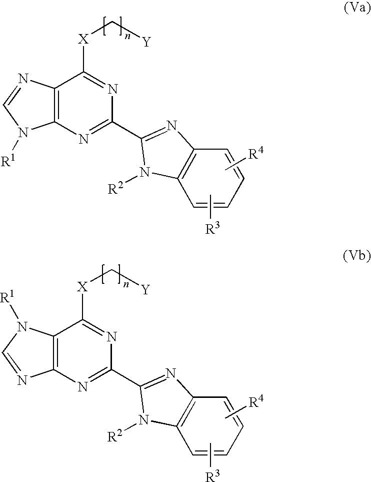

a stereoisomer thereof or a mixture of its stereoisomers, an N-oxide thereof, a prodrug thereof, or a pharmaceutically acceptable salt thereof, wherein n, X, Y, R¹, R², R³ and R⁴ are as defined above.

In another embodiment the derivative of the invention is a purinyl-pyridinyl derivative of Formula VIa or VIb

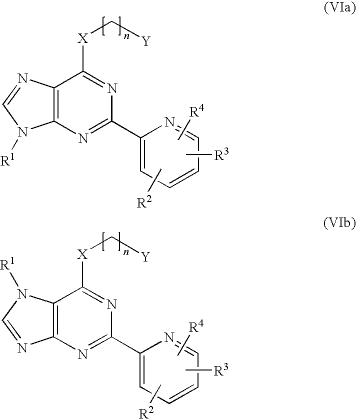

a stereoisomer thereof or a mixture of its stereoisomers, an N-oxide thereof, a prodrug thereof, or a pharmaceutically acceptable salt thereof, wherein n, X, Y, R¹, R², R³ and R⁴ are as defined above.

In another embodiment the derivative of the invention is a compound of Formula Ia, Ib, IIa, IIb, IIIa, IIIb, IVa, IVb, Va, Vb, VIa or VIb, wherein n is 0, 1, 2 or 3.

In another embodiment n is 0, 1 or 2.

In another embodiment n is 0 or 1.

In another embodiment n is 0.

In another embodiment n is 1.

In another embodiment n is 2.

In another embodiment the derivative of the invention is a compound of formula Ia, Ib, IIa, IIb, IIIa, IIIb, IVa, IVb, Va, Vb, VIa or VIb, wherein X represents O, S or NR′; wherein R′ represents hydrogen, alkyl, cycloalkyl, phenyl or benzyl.

In another embodiment X represents NR′; wherein R′ represents hydrogen, alkyl or cycloalkyl.

In another embodiment X represents NR′; wherein R′ represents hydrogen or methyl.

In another embodiment X represents O, S or NH.

In another embodiment X represents O.

In another embodiment X represents S.

In another embodiment X represents NH.

In another embodiment the derivative of the invention is a compound of Formula Ia, Ib, IIa, IIb, IIIa, IIIb, IVa, IVb, Va, Vb, VIa or VIb, wherein Y represents alkyl, cycloalkyl or phenyl; which alkyl, cycloalkyl and phenyl are optionally substituted with one substituent selected from the group consisting of alkyl, cycloalkyl, halo, trifluoromethyl, trifluoromethoxy, hydroxy, alkoxy, cyano, nitro and amino.

In another embodiment Y represents cycloalkyl or phenyl; which phenyl is optionally substituted with one substituent selected from the group consisting of alkyl, cycloalkyl, halo, trifluoromethyl, trifluoromethoxy, hydroxy, alkoxy, cyano, nitro and amino.

In another embodiment Y represents cycloalkyl or phenyl; which phenyl is optionally substituted with one substituent selected from the group consisting of halo, in particular fluoro or chloro, or trifluoromethyl.

In another embodiment Y represents cycloalkyl or phenyl; which phenyl is optionally substituted with one halo, in particular fluoro, chloro or bromo.

In another embodiment Y represents cycloalkyl, e.g. cyclopropyl, cyclobutyl, cyclopentyl, cyclohexyl or cycloheptyl.

In another embodiment Y represents cyclohexyl.

In another embodiment Y represents phenyl; which phenyl is optionally substituted with one substituent selected from the group consisting of halo, trifluoromethyl, trifluoromethoxy cyano, nitro or amino.

In another embodiment Y represents phenyl; which phenyl is optionally substituted with one substituent selected from the group consisting of halo, trifluoromethyl, cyano, nitro or amino.

In another embodiment Y represents phenyl; which phenyl is optionally substituted with one halo, in particular fluoro, chloro or bromo.

In another embodiment Y represents phenyl; which phenyl is substituted with one fluoro.

In another embodiment Y represents phenyl; which phenyl is substituted with one chloro.

In another embodiment Y represents phenyl; which phenyl is optionally substituted with one trifluoromethyl.

In another embodiment Y represents phenyl; which phenyl is optionally substituted with one cyano.

In another embodiment Y represents phenyl; which phenyl is optionally substituted with one nitro.

In another embodiment Y represents phenyl; which phenyl is optionally substituted with one amino.

In another embodiment Y represents phenyl.

In another embodiment the derivative of the invention is a compound of Formula Ia, Ib, IIa, IIb, IIIa, IIIb, IVa, IVb, Va, Vb, VIa or VIb, wherein R¹ represents hydrogen, alkyl or alkoxy-alkyl.

In another embodiment R¹ represents hydrogen.

In another embodiment R¹ represents alkyl.

In another embodiment R¹ represents methyl.

In another embodiment R¹ represents ethyl.

In another embodiment R¹ represents alkoxy-alkyl.

In another embodiment R¹ represents methoxy-ethyl.

In another embodiment the derivative of the invention is a compound of formula Ia or Ib, wherein Het represents a heterocyclic group selected from pyrazolyl, imidazolyl, indazolyl, benzimidazolyl, pyridinyl and cyclopentapyrazolyl, which pyrazolyl, imidazolyl, indazolyl, benzimidazolyl, pyridinyl and cyclopentapyrazolyl may optionally be substituted with one substituent selected from the group consisting of alkyl, hydroxy-alkyl, cycloalkyl, cycloalkyl-alkyl, alkenyl, alkynyl, halo, trifluoromethyl, trifluoromethoxy, hydroxy, alkoxy, alkoxy-carbonyl, carboxy, cyano, nitro, amino, amino-carbonyl, N,N-dialkyl-amino-carbonyl, phenyl and benzyl.

In another embodiment the derivative of the invention is a compound of formula Ia or Ib, wherein Het represents a heterocyclic group selected from pyrazolyl, pyridinyl and cyclopentapyrazolyl, which pyrazolyl, pyridinyl and cyclopentapyrazolyl may optionally be substituted with one substituent selected from the group consisting of alkyl, hydroxy-alkyl, halo, trifluoromethyl, alkoxy-carbonyl, nitro, amino and phenyl.

In another embodiment the derivative of the invention is a compound of formula Ia or Ib, wherein Het represents pyrazolyl optionally substituted one time with alkyl.

In another embodiment the derivative of the invention is a compound of formula Ia or Ib, wherein Het represents pyrazolyl optionally substituted one time with methyl.

In another embodiment the derivative of the invention is a compound of formula Ia or Ib, wherein Het represents pyrazolyl substituted one time with alkyl.

In another embodiment the derivative of the invention is a compound of formula Ia or Ib, wherein Het represents pyrazolyl substituted one time with methyl.

In another embodiment the derivative of the invention is a compound of formula Ia or Ib, wherein Het represents pyrazolyl optionally substituted one time with halo.

In another embodiment the derivative of the invention is a compound of formula Ia or Ib, wherein Het represents pyrazolyl substituted one time with halo.

In another embodiment the derivative of the invention is a compound of formula Ia or Ib, wherein Het represents pyrazolyl optionally substituted one time with trifluoromethyl.

In another embodiment the derivative of the invention is a compound of formula Ia or Ib, wherein Het represents pyrazolyl substituted one time with trifluoromethyl.

In another embodiment the derivative of the invention is a compound of formula Ia or Ib, wherein Het represents pyrazolyl optionally substituted one time with nitro.

In another embodiment the derivative of the invention is a compound of formula Ia or Ib, wherein Het represents pyrazolyl substituted one time with nitro.

In another embodiment the derivative of the invention is a compound of formula Ia or Ib, wherein Het represents pyrazolyl optionally substituted one time with amino.

In another embodiment the derivative of the invention is a compound of formula Ia or Ib, wherein Het represents pyrazolyl substituted one time with amino.

In another embodiment the derivative of the invention is a compound of formula Ia or Ib, wherein Het represents pyrazolyl optionally substituted one time with phenyl.

In another embodiment the derivative of the invention is a compound of formula Ia or Ib, wherein Het represents pyrazolyl.

In another embodiment the derivative of the invention is a compound of formula Ia or Ib, wherein Het represents pyridinyl.

In another embodiment the derivative of the invention is a compound of formula Ia or Ib, wherein Het represents cyclopentapyrazolyl, optionally substituted one time with alkyl.

In another embodiment the derivative of the invention is a compound of formula Ia or Ib, wherein Het represents cyclopentapyrazolyl, optionally substituted one time with methyl.

In another embodiment the derivative of the invention is a compound of Formula IIa, IIb, IIIa, IIIb, IVa, IVb, Va, Vb, VIa or VIb, wherein two of R², R³ and R⁴ represent hydrogen; and the remaining one of R², R³ and R⁴ represents alkyl, hydroxy-alkyl, cycloalkyl, cycloalkyl-alkyl, alkenyl, alkynyl, halo, trifluoromethyl, trifluoromethoxy, hydroxy, alkoxy, alkoxy-carbonyl, carboxy, cyano, nitro, amino, amino-carbonyl, N,N-dialkyl-amino-carbonyl, phenyl or benzyl.

In another embodiment two of R², R³ and R⁴ represent hydrogen; and the remaining one of R², R³ and R⁴ represents alkyl, hydroxy-alkyl, halo, trifluoromethyl, alkoxy-carbonyl, nitro, amino or phenyl.

In another embodiment two of R², R³ and R⁴ represent hydrogen; and the remaining one of R², R³ and R⁴ represents alkyl.

In another embodiment two of R², R³ and R⁴ represent hydrogen; and the remaining one of R², R³ and R⁴ represents methyl.

In another embodiment two of R², R³ and R⁴ represent hydrogen; and the remaining one of R², R³ and R⁴ represents halo.

In another embodiment two of R², R³ and R⁴ represent hydrogen; and the remaining one of R², R³ and R⁴ represents trifluoromethyl.

In another embodiment two of R², R³ and R⁴ represent hydrogen; and the remaining one of R², R³ and R⁴ represents nitro.

In another embodiment two of R², R³ and R⁴ represent hydrogen; and the remaining one of R², R³ and R⁴ represents trifluoromethyl.

In another embodiment two of R², R³ and R⁴ represent hydrogen; and the remaining one of R², R³ and R⁴ represents amino.

In another embodiment R², R³ and R⁴ represent hydrogen.

In another embodiment of the invention alkyl represents methyl.

In another embodiment of the invention alkyl represents ethyl.

In another embodiment of the invention halo represents fluoro.

In another embodiment of the invention halo represents chloro.

In another embodiment the derivative of the invention is:
(4-Chloro-phenyl)-[9-methyl-2-(3-trifluoromethyl-pyrazol-1-yl)-9H-purin-6-yl]-amine;
(4-Chloro-phenyl)-(9-methyl-2-pyrazol-1-yl-9H-purin-6-yl)-amine;
Cyclohexyl-(9-methyl-2-pyrazol-1-yl-9H-purin-6-yl)-amine;
(4-Chloro-phenyl)-[2-(4-chloro-pyrazol-1-yl)-9-methyl-9H-purin-6-yl]-amine;
(4-Chloro-phenyl)-[9-methyl-2-(4-methyl-pyrazol-1-yl)-9H-purin-6-yl]-amine;
(4-Fluoro-phenyl)-[9-methyl-2-(3-methyl-pyrazol-1-yl)-9H-purin-6-yl]-amine;
(4-Chloro-phenyl)-[9-methyl-2-(3-nitro-pyrazol-1-yl)-9H-purin-6-yl]-amine;
[2-(3-Amino-pyrazol-1-yl)-9-methyl-9H-purin-6-yl]-(4-chloro-phenyl)-amine;
(4-Chloro-phenyl)-[9-methyl-2-(3-methyl-pyrazol-1-yl)-9H-purin-6-yl]-amine;
[9-Methyl-2-(3-methyl-pyrazol-1-yl)-9H-purin-6-yl]-phenyl-amine;
[9-Methyl-2-(3-methyl-pyrazol-1-yl)-9H-purin-6-yl]-phenethyl-amine;
6-(4-Chloro-phenoxy)-9-methyl-2-(3-methyl-pyrazol-1-yl)-9H-purine; (4-Fluoro-phenyl)-[9-methyl-2-(3-methyl-5,6-dihydro-4H-cyclopentapyrazol-2-yl)-9H-purin-6-yl]-amine;
(4-Chloro-phenyl)-(9-methyl-2-pyridin-2-yl-9H-purin-6-yl)-amine;
Cyclohexyl-(9-methyl-2-pyridin-2-yl-9H-purin-6-yl)-amine; or
   a stereoisomer thereof or a mixture of its stereoisomers, an N-oxide thereof, a prodrug thereof, or a pharmaceutically acceptable salt thereof.

Any combination of two or more of the embodiments described herein is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contain of from one to eighteen carbon atoms (C₁₋₁₈-alkyl), e.g. from one to six carbon atoms (C₁₋₆-alkyl; lower alkyl), including pentyl, isopentyl, neopentyl, tertiary pentyl, hexyl and isohexyl. In another embodiment alkyl represents a C₁₋₄-alkyl group, including butyl, isobutyl, secondary butyl, and tertiary butyl. In another embodiment of this invention alkyl represents a C₁₋₃-alkyl group, which may in particular be methyl, ethyl, propyl or isopropyl.

In the context of this invention an alkenyl group designates a carbon chain containing one or more double bonds, including di-enes, tri-enes and poly-enes. In another embodiment the alkenyl group of the invention comprises of from two to eight carbon atoms (C₂₋₈-alkenyl), e.g. from two to six carbon atoms (C₂₋₆-alkenyl), including at least one double bond. In another embodiment the alkenyl group of the invention is ethenyl; 1- or 2-propenyl; 1-, 2- or 3-butenyl, or 1,3-butenyl; 1-, 2-, 3-, 4- or 5-hexenyl, or 1,3-hexenyl, or 1,3,5-hexenyl; 1-, 2-, 3-, 4-, 5-, 6-, or 7-octenyl, or 1,3-octenyl, or 1,3,5-octenyl, or 1,3,5,7-octenyl.

In the context of this invention an alkynyl group designates a straight or branched carbon chain containing one or more triple bonds, including di-ynes, tri-ynes and poly-ynes. In another embodiment the alkynyl group of the invention comprises of from two to eight carbon atoms (C₂₋₈-alkynyl), e.g. from two to six carbon atoms (C₂₋₆-alkynyl), including at least one triple bond. In another embodiment the alkynyl group of the invention is ethynyl; 1-, or 2-propynyl; 1-, 2-, or 3-butynyl, or 1,3-butadiynyl; 1-, 2-, 3-, 4-pentynyl, or 1,3-pentadiynyl; 1-, 2-, 3-, 4-, or 5-hexynyl, or 1,3-hexadiynyl or 1,3,5-hexatriynyl; 1-, 2-, 3-, 4-, 5- or 6-heptynyl, or 1,3-heptdiynyl, or 1,3,5-hepttriynyl; 1-, 2-, 3-, 4-, 5-, 6- or 7-octynyl, or 1,3-octdiynyl, or 1,3,5-octtriynyl, or 1,3,5,7-octtetraynyl.

In the context of this invention a hydroxy-alkyl group designates an alkyl group as defined above, which hydroxy-alkyl group is substituted with one or more hydroxy groups. Examples of hydroxy-alkyl groups of the invention include 2-hydroxy-ethyl, 3-hydroxy-propyl, 4-hydroxy-butyl, 5-hydroxy-pentyl and 6-hydroxy-hexyl.

In the context of this invention a cycloalkyl group designates a cyclic alkyl group, preferably containing of from three to ten carbon atoms (C₃₋₁₀-cycloalkyl), e.g. from three to eight carbon atoms (C₃₋₈-cycloalkyl), including cyclopropyl, cyclobutyl, cyclopentyl, cyclohexyl, cycloheptyl and cyclooctyl; or e.g. from three to six carbon atoms (C₃₋₆-cycloalkyl), including cyclopropyl, cyclobutyl, cyclopentyl and cyclohexyl.

In the context of this invention a cycloalkyl-alkyl group designates a cycloalkyl group as defined above, which cycloalkyl group is substituted on an alkyl group as also defined above. Examples of cycloalkyl-alkyl groups of the invention include cyclopropylmethyl and cyclopropylethyl.

In the context of this invention an alkoxy group designates an “alkyl-O-”group, wherein alkyl is as defined above. Examples of alkoxy groups of the invention include methoxy and ethoxy.

In the context of this invention an alkoxy-alkyl group designates an “alkyl-O-alkyl-”group, wherein alkyl is as defined above. Examples of alkoxy-alkyl groups of the invention include methoxy-methyl, methoxy-ethyl, ethoxy-methyl, and ethoxy-ethyl.

In the context of this invention an alkoxy-carbonyl group designates an “alkyl-O—CO- ”group, wherein alkyl is as defined above. Examples of alkoxy-carbonyl groups of the invention include the methyl-, ethyl- and propyl-ester group.

In the context of this invention an amino-carbonyl group designates an “amino-CO- ”group.

In the context of this invention an N,N-dialkyl-amino-carbonyl group designates a (tertiary) amino-carbonyl group, disubstituted with alkyl groups as defined above.

### Isomers

The derivatives of the present invention may exist in different stereoisomeric forms, including enantiomers, diastereomers, as well as geometric isomers (cis-trans isomers). The invention includes all such isomers and any mixtures thereof including racemic mixtures.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystallisation of d- or l- (tartrates, mandelates, or camphorsulphonate) salts for example.

The chemical compounds of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Moreover, some of the chemical compounds of the invention being oximes, may thus exist in two forms, syn- and anti-form (Z- and E-form), depending on the arrangement of the substituents around the —C═N— double bond. A chemical compound of the present invention may thus be the syn- or the anti-form (Z- and E-form), or it may be a mixture hereof.

### Pharmaceutically Acceptable Salts

The derivatives of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulfonate derived from benzensulfonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulfonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a derivative of the invention and its pharmaceutically acceptable acid addition salt.

Metal salts of a chemical compound of the invention include alkali metal salts, such as the sodium salt of a chemical compound of the invention containing a carboxy group.

In the context of this invention the “onium salts” of N-containing compounds are also contemplated as pharmaceutically acceptable salts. Examples of “onium salts” include the alkyl-onium salts, the cycloalkyl-onium salts, and the cycloalkylalkyl-onium salts.

The derivative of the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

### Methods of Preparation

The derivatives of the invention may be prepared by conventional methods of chemical synthesis, e.g. those described in the working examples. The starting materials for the processes described in the present application are known or may readily be prepared by conventional methods from commercially available chemicals.

The end products of the reactions described herein may be isolated by conventional techniques, e.g. by extraction, crystallisation, distillation, chromatography, etc.

### Biological Activity

The derivatives of the invention have been subjected to in vitro experiments and found useful as potassium channel modulating agents. The compounds of the invention are capable of selectively modulating SK1, SK2 and/or SK3 channels.

Therefore, in another aspect, the invention relates to the use of the derivatives of the invention for the manufacture of medicaments, which medicament may be useful for the treatment or alleviation of a disease or a disorder associated with the activity of potassium channels, e.g. SK channels, e.g. SK1, SK2 and/or SK3 channels.

In another embodiment, the disease or a disorder associated with the activity of potassium channels is a respiratory disease, epilepsy, convulsions, seizures, absence seizures, vascular spasms, coronary artery spasms, renal disorders, polycystic kidney disease, bladder spasms, overactive bladder (OAB), urinary incontinence, bladder outflow obstruction, interstitiel cystitis (IC), erectile dysfunction, gastrointestinal dysfunction, secretory diarrhoea, ischaemia, cerebral ischaemia, ischaemic heart disease, angina pectoris, coronary heart disease, autism, ataxia, traumatic brain injury, Parkinson's disease, bipolar disorder, psychosis, schizophrenia, anxiety, depression, mania, mood disorders, dementia, memory and attention deficits, Alzheimer's disease, amyotrophic lateral sclerosis (ALS), dysmenorrhea, narcolepsy, Reynaud's disease, intermittent claudication, Sjogren's syndrome, arrhythmia, hypertension, myotonic muscle dystrophia, spasticity, xerostomi, diabetes type II, hyperinsulinemia, premature labour, baldness, cancer, irritable bowel syndrome (IBS), immune suppression, migraine or pain, e.g. pelvic pain or abdominal pain, or withdrawal symptoms caused by the termination of abuse of chemical substances, in particular opioids, heroin, cocaine and morphine, benzodiazepines and benzodiazepine-like drugs, and alcohol.

In another embodiment the disease or a disorder associated with the activity of potassium channels is a respiratory disease, urinary incontinence, erectile dysfunction, anxiety, epilepsy, psychosis, schizophrenia, amyotrophic lateral sclerosis (ALS) or pain.

In another embodiment the disease or a disorder associated with the activity of potassium channels is a respiratory disease, in particular asthma, cystic fibrosis, chronic obstructive pulmonary disease (COPD) or rhinorrhea.

In another embodiment the disease or a disorder associated with the activity of potassium channels is overactive bladder, e.g. urinary incontinence.

In another embodiment the disease or a disorder associated with the activity of potassium channels is epilepsy, seizures, absence seizures or convulsions.

In another embodiment the disease or a disorder associated with the activity of potassium channels is schizophrenia.

In another embodiment the disease or a disorder associated with the activity of potassium channels is pain.

The compounds tested showed a biological activity determined as described herein in the micromolar and sub-micromolar range, i.e. of from below 1 to above 100 μM e.g. from below 0.1 to about 10 μM.

### Pharmaceutical Compositions

In yet another aspect the invention provides novel pharmaceutical compositions comprising a therapeutically effective amount of the derivatives of the invention.

While a derivative of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers and/or diluents.

In another embodiment, the invention provides pharmaceutical compositions comprising the derivative of the invention, or a pharmaceutically acceptable salt or derivative thereof, together with one or more pharmaceutically acceptable carriers therefore and, optionally, other therapeutic and/or prophylactic ingredients. The carrier(s) must be “acceptable” in the sense of being compatible with the other ingredients of the formulation and not deleterious to the recipient thereof.

Pharmaceutical compositions of the invention may be those suitable for oral, rectal, bronchial, nasal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The derivates of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The derivative of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The derivative according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilisers, buffers, artificial and natural sweeteners, dispersants, thickeners, solubilizing agents, and the like.

For topical administration to the epidermis the chemical compound according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form. In the latter case of a dropper or pipette, this may be achieved by the patient administering an appropriate, predetermined volume of the solution or suspension. In the case of a spray, this may be achieved for example by means of a metering atomising spray pump.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

In one embodiment, the invention provides tablets or capsules for oral administration

In another embodiment, the invention provides and liquids for intravenous administration and continuous infusion.

Further details on techniques for formulation and administration may be found in the latest edition of Remington's Pharmaceutical Sciences (Maack Publishing Co., Easton, Pa.).

A therapeutically effective dose refers to that amount of active ingredient which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions which exhibit large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

The actual dosage depends on the nature and severity of the disease being treated and the route of administration, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, e.g. from about 1 to about 100 mg, e.g. from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Other ranges are from about 0.1 μg/kg to about 10 mg/kg/day i.v., and from about 1 μg/kg to about 100 mg/kg/day p.o.

### Methods of Therapy

In another aspect the invention provides a method for the prevention, treatment or alleviation of a disease or a disorder or a condition of a living animal body, including a human, which disease, disorder or condition is responsive to modulation of potassium channels, in particular SK channels, and which method comprises comprising administering to such a living animal body, including a human, in need thereof a therapeutically-effective amount of a derivative of the invention.

The indications contemplated according to the invention are those stated above.

It is at present contemplated that suitable dosage ranges are 0.1 to 1000 milligrams daily, 10-500 milligrams daily, or 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Other ranges are from about 0.001 to about 1 mg/kg i.v. and from about 0.1 to about 10 mg/kg p.o.

### EXAMPLES

The invention is further illustrated with reference to the following examples, which are not intended to be in any way limiting to the scope of the invention as claimed.

### Example 1

### 2,6-Dichloro-9-methyl-9H-purine and 2,6-Dichloro-7-methyl-7H-purine (Intermediate compounds)

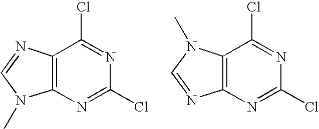

Sodium hydride (60% in mineral oil, 2.53 g, 63.5 mmol) was added to an ice-cooled solution of 2,6-dichloropurine (10.0 g, 52.9 mmol) in tetrahydrofuran (75 mL) and the mixture was stirred for 30 min. Methyl iodide (3.29 mL, 52.9 mmol) was added drop-wise and the reaction mixture was stirred at room temperature over night. Water was added and the aqueous phase was extracted with ethyl acetate. The combined organic phases were dried over magnesium sulphate, filtered and concentrated in vacuo. Dichloromethane was added and undissolved material collected by filtration. The crystalline compound turned out to be 2,6-dichloro-7-methyl-7H-purine (1.19 g, 11%) The filtrate was concentrated in vacuo and purified by flash chromatography (ethyl acetate/hepatane) to give 2,6-dichloro-9-methyl-9H-purine (3.0 g, 28%).

### Example 2

### N-(4-Chloro-phenyl)-formamide

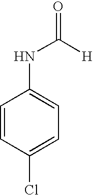

4-Chloroaniline (15 g, 117 mmol) and formic acid (25 mL, 663 mL) were heated to reflux for 2 hours. The mixture was concentrated in vacuo. Saturated aqueous sodium hydrogencarbonate was added and the aqueous phase was extracted twice with ethyl acetate. The combined organic phases were dried over magnesium sulphate, filtered and concentrated in vacuo to give N-(4-chloro-phenyl)-formamide (17.6 g, 97%) as a grey crystalline compound.

### N-(4-Fluoro-phenyl)-formamide

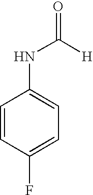

Was prepared according to Example 2 from 4-fluoroaniline and formic acid.

### N-Phenyl-formamide

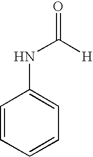

Was prepared according to Example 2 from aniline and formic acid.

### Example 3

### (2-Chloro-9-methyl-9H-purin-6-yl)-(4-chloro-phenyl)-amine (Intermediate compound)

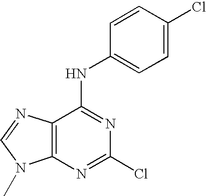

N-(4-Chloro-phenyl)-formamide (766 mg, 4.93 mmol) was dissolved in N,N-dimethylformamide (10 mL). Sodium hydride (60% in mineral oil, 240 mg, 5.91 mmol) was added and the mixture was stirred for 30 min. 2,6-Dichloro-9-methyl-9H-purine (1.0 g, 4.93 mmol) was added and the reaction mixture was heated at 80° C. for 2 hours cooled to room temperature and poured into water. The resulting precipitate was collected by filtration, washed with water and dried to give (2-chloro-9-methyl-9H-purin-6-yl)-(4-chloro-phenyl)-amine (1.2 g, 4.08 mmol, 83%).

### (2-Chloro-9-methyl-9H-purin-6-yl)-(4-fluoro-phenyl)-amine

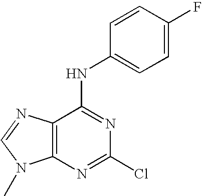

Was prepared according to Example 3 from N-(4-fluoro-phenyl)-formamide and 2,6-dichloro-9-methyl-9H-purine.

### (2-Chloro-9-methyl-9H-purin-6-yl)-phenyl-amine

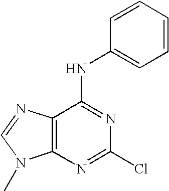

Was prepared according to Example 3 from N-phenyl-formamide and 2,6-dichloro-9-methyl-9H-purine.

### 2-Chloro-6-(4-chloro-phenoxy)-9-methyl-9H-purine

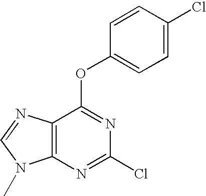

Was prepared according to Example 3 from 4-chlorophenol and 2,6-dichloro-9-methyl-9H-purine.

### Example 4

### (2-Chloro-9-methyl-9H-purin-6-yl)-cyclohexyl-amine (Intermediate compound)

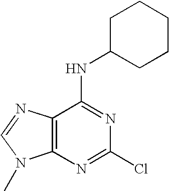

2,6-Dichloro-9-methyl-9H-purine (1.40 g, 6.93 mmol) was dissolved in acetonitrile (25 mL). Triethylamine (4.81 mL, 34.5 mL) and cyclohexylamine (0.79 mL, 6.90 mmol) were added and the reaction mixture was stirred at 50° C. over night. The reaction mixture was cooled to room temperature and concentrated in vacuo. Water was added followed by extraction with ethyl acetate. The combined organic phases were dried over magnesium sulphate, filtered and concentrated in vacuo to give (2-chloro-9-methyl-9H-purin-6-yl)-cyclohexyl-amine (1.5 g, 82%) as a crystalline compound.

### Example 5

### (2-Chloro-9-methyl-9H-purin-6-yl)-phenethyl-amine

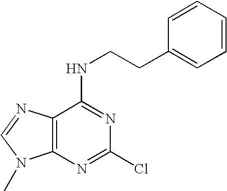

Was prepared according to Example 4 from 9-methyl-2,6-dichloro-9H-purine and phenethylamine.

### Example 6

### (4-Chloro-phenyl)-[9-methyl-2-(3-trifluoromethyl-pyrazol-1-yl)-9H-purin-6-yl]-amine (Compound 6.1)

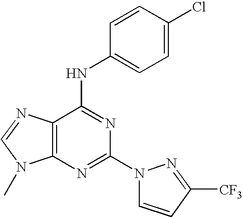

Sodium hydride (60% in mineral oil, 195 mg, 4.9 mmol) was added to 3-(trifluoromethyl)pyrazole (555 mg, 4.1 mmol) dissolved N,N-dimethylformamide (10 mL) and the mixture was stirred for 30 min. (2-Chloro-9-methyl-9H-purin-6-yl)-(4-chloro-phenyl)-amine (1.2 g 4.08 mmol) was added and the reaction mixture was heated at 100° C. over night. Water was added and the resulting white solid was collected by filtration. Recrystallisation from ethyl acetate gave (4-chloro-phenyl)-[9-methyl-2-(3-trifluoromethyl-pyrazol-1-yl)-9H-purin-6-yl]-amine as a white crystalline compound.

LC-ESI-HRMS of [M+H]+ shows 394.0794 Da. Calc. 394.07948 Da, dev. −0.2 ppm.

### (4-Chloro-phenyl)-(9-methyl-2-pyrazol-1-yl-9H-purin-6-yl)-amine (Compound 6.2)

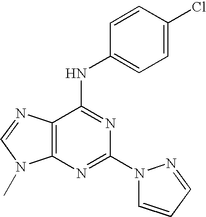

Was prepared according to Example 6 from (2-chloro-9-methyl-9H-purin-6-yl)-(4-chloro-phenyl)-amine and pyrazole.

LC-ESI-HRMS of [M+H]+ shows 326.0924 Da. Calc. 326.092096 Da, dev. 0.9 ppm.

### Cyclohexyl-(9-methyl-2-pyrazol-1-yl-9H-purin-6-yl)-amine (Compound 6.3)

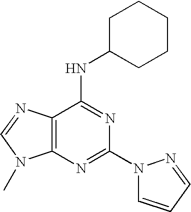

Was prepared according to Example 6 from (2-chloro-9-methyl-9H-purin-6-yl)-cyclohexyl-amine and pyrazole.

LC-ESI-HRMS of [M+H]+ shows 298.1792 Da. Calc. 298.178018 Da, dev. 4 ppm.

### (4-Chloro-phenyl)-[2-(4-chloro-pyrazol-1-yl)-9-methyl-9H-purin-6-yl]-amine (Compound 6.4)

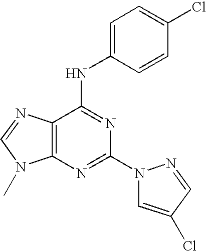

Was prepared according to Example 6 from (2-chloro-9-methyl-9H-purin-6-yl)-(4-chloro-phenyl)-amine and 4-chloro-1H-pyrazole.

LC-ESI-HRMS of [M+H]+ shows 360.0516 Da. Calc. 360.053124 Da, dev. −4.2 ppm.

### (4-Chloro-phenyl)-[9-methyl-2-(4-methyl-pyrazol-1-yl)-9H-purin-6-yl]-amine (Compound 6.5)

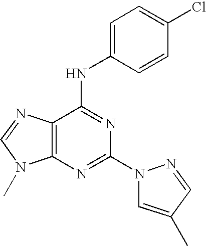

Was prepared according to Example 6 from (2-chloro-9-methyl-9H-purin-6-yl)-(4-chloro-phenyl)-amine and 4-methylpyrazole.

LC-ESI-HRMS of [M+H]+ shows 340.1073 Da. Calc. 340.107746 Da, dev. −1.3 ppm.

### (4-Fluoro-phenyl)-[9-methyl-2-(3-methyl-pyrazol-1-yl)-9H-purin-6-yl]-amine (Compound 6.6)

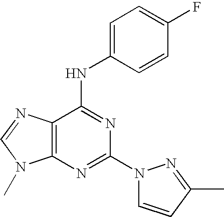

Was prepared according to Example 6 from (2-chloro-9-methyl-9H-purin-6-yl)-(4-fluoro-phenyl)-amine and 3-methylpyrazole.

LC-ESI-HRMS of [M+H]+ shows 324.1377 Da. Calc. 324.137296 Da, dev. 1.2 ppm.

### (4-Chloro-phenyl)-[9-methyl-2-(3-nitro-pyrazol-1-yl)-9H-purin-6-yl]-amine (Compound 6.7)

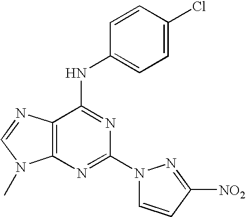

Was prepared according to Example 6 from (2-chloro-9-methyl-9H-purin-6-yl)-(4-chloro-phenyl)-amine and 5-nitro-1H-pyrazole.

LC-ESI-HRMS of [M+H]+ shows 371.0777 Da. Calc. 371.077175 Da, dev. 1.4 ppm.

### Example 7

### [2-(3-Amino-pyrazol-1-yl)-9-methyl-9H-purin-6-yl]-(4-chloro-phenyl)-amine (Compound 7.1)

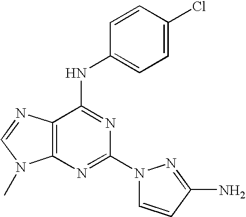

(4-Chloro-phenyl)-[9-methyl-2-(3-nitro-pyrazol-1-yl)-9H-purin-6-yl]-amine (2.3 g, 6.2 mmol) was dissolved in methanol (20 mL) and dichloromethane (20 mL). Raney nickel was added and the mixture was stirred under a hydrogen atmosphere for two days. Tetrahydrofuran was added. Filtration through celite followed by concentration in vacuo gave [2-(3amino-pyrazol-1-yl)-9-methyl-9H-purin-6-yl]-(4-chloro-phenyl)-amine (1.7 g, 80%) as a green solid.

LC-ESI-HRMS of [M+H]+ shows 341.1015 Da. Calc. 341.102995 Da, dev. −4.4 ppm

### Example 8

### (4-Chloro-phenyl)-(2-hydrazino-9-methyl-9H-purin-6-yl)-amine

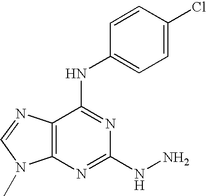

(2-Chloro-9-methyl-9H-purin-6-yl)-(4-chloro-phenyl)-amine (3.58 g, 12.1 mmol) was dissolved in tetrahydrofuran (50 mL). Hydrazine monohydrate (26 mL, 536 mmol) was added and the reaction mixture was heated to reflux over night. Water was added and the resulting solid was collected by filtration, washed with water and dried to give (4-chloro-phenyl)-(2-hydrazino-9-methyl-9H-purin-6-yl)-amine (3.16 g, 90%) as a white crystalline compound.

### (4-Fluoro-phenyl)-(2-hydrazino-9-methyl-9H-purin-6-yl)-amine

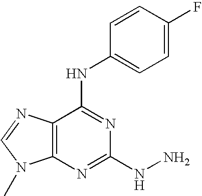

Was prepared according to Example 8 from (2-chloro-9-methyl-9H-purin-6-yl)-(4-fluoro-phenyl)-amine and hydrazine monohydrate.

### (2-Hydrazino-9-methyl-9H-purin-6-yl)-phenyl-amine

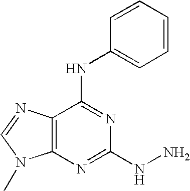

Was prepared according to Example 8 from (2-chloro-9-methyl-9H-purin-6-yl)-phenyl-amine and hydrazine monohydrate.

### (2-Hydrazino-9-methyl-9H-purin-6-yl)-phenethyl-amine

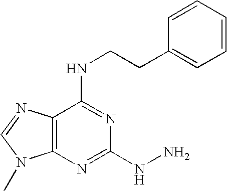

Was prepared according to Example 8 from (2-chloro-9-methyl-9H-purin-6-yl)-phenethyl-amine and hydrazine monohydrate.

### [6-(4-Chloro-phenoxy)-9-methyl-9H-purin-2-yl]-hydrazine

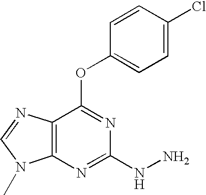

Was prepared according to Example 8 from 2-chloro-6-(4-chloro-phenoxy)-9-methyl-9H-purine and hydrazine monohydrate.

### Example 9

### (4-Chloro-phenyl)-[9-methyl-2-(3-methyl-pyrazol-1-yl)-9H-purin-6-yl]-amine (Compound 9.1)

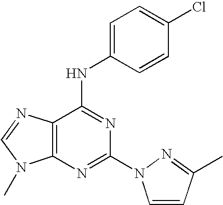

(4-Chloro-phenyl)-(2-hydrazino-9-methyl-9H-purin-6-yl)-amine (4.74 g, 16.36 mmol) and acetylacetaldehyde dimethylacetal (2.5 mL, 18.84 mmol) in ethanol (50 mL) were heated to reflux for 20 min. Water was added and the white crystalline compound was collected by filtration, washed with water and dried to give (4-chlorophenyl)-[9-methyl-2-(3-methyl-pyrazol-1-yl)-9H-purin-6-yl]-amine (3.7 g, 67%). LC-ESI-HRMS of [M+H]+ shows 340.1095 Da. Calc. 340.107746 Da, dev. 5.2 ppm.

### [9-Methyl-2-(3-methyl-pyrazol-1-yl)-9H-purin-6-yl]-phenyl-amine (Compound 9.2)

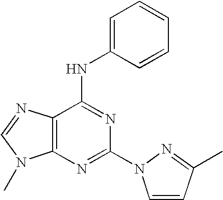

Was prepared according to Example 9 from (2-hydrazino-9-methyl-9H-purin-6-yl)-phenyl-amine and acetylacetaldehyde dimethylacetal.

LC-ESI-HRMS of [M+H]+ shows 306.1476 Da. Calc. 306.146718 Da, dev. 2.9 ppm.

### [9-Methyl-2-(3-methyl-pyrazol-1-yl)-9H-purin-6-yl]-phenethyl-amine (Compound 9.3)

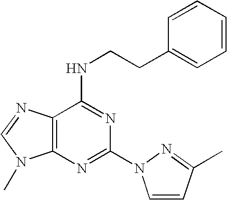

Was prepared according to Example 9 from (2-hydrazino-9-methyl-9H-purin-6-yl)-phenethyl-amine and acetylacetaldehyde dimethylacetal.

LC-ESI-HRMS of [M+H]+ shows 334.179 Da. Calc. 334.178018 Da, dev. 2.9 ppm.

### 6-(4-Chloro-phenoxy)-9-methyl-2-(3-methyl-pyrazol-1-yl)-9H-purine (Compound 9.4)

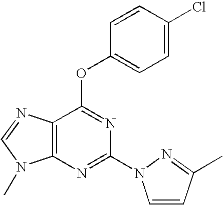

Was prepared according to Example 9 from [6-(4-chloro-phenoxy)-9-methyl-9H-purin-2-yl]-hydrazine and acetylacetaldehyde dimethylacetal.

### (4-Fluoro-phenyl)-[9-methyl-2-(3-methyl-5,6-dihydro-4H-cyclopentapyrazol-2-yl)-9H-purin-6-yl]-amine (Compound 9.5)

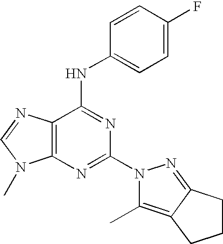

Was prepared according to Example 9 from (4-fluoro-phenyl)-(2-hydrazino-9-methyl-9H-purin-6-yl)-amine and 2-acetylcyclopentanone.

LC-ESI-HRMS of [M+H]+ shows 364.1699 Da. Calc. 364.168596 Da, dev. 3.6 ppm.

### Example 10

### 5-Amino-1-methyl-1H-imidazole-4-carboxylic acid amide (Intermediate compound)

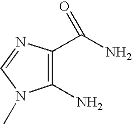

Potassium hydroxide (3.45 g, 61.5 mmol) was added to an ice cooled solution of 4-aminoimidazole-5-carboxamide hydrochloride (5.0 g, 30.8 mmol) in N,N-dimethylformamide (50 mL) and stirred for 3 hours at 0° C. Methyl iodide (1.91 mL, 30.8 mmol) was added and the mixture was stirred over night at 0° C. Filtration followed by wash with methanol and evaporation of the organic phase gave 5-amino-1-methyl-1H-imidazole-4-carboxylic acid amide (2.2 g, 51%) as a brown solid.

### Example 11

### 9-Methyl-2-pyridin-2-yl-9H-purin-6-ol (Intermediate compound)

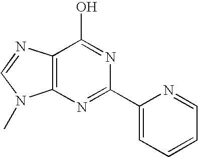

5-Amino-1-methyl-1H-imidazole-4-carboxylic acid amide (2.2 g, 15.7 mmol) was added to a mixture of 2-picolinic acid (1.93 g, 15.7 mmol) and triethylamine (15.3 mL, 110 mmol) in dichloromethane (20 mL). The mixture was cooled to 0° C. and 1-propanephosphoric acid cyclic anhydride (14.0 mL, 23.5 mmol) was added dropwise. Stirring was continued at 0° C. for 30 min. and at room temperature over night. The mixture was acidified with aqueous hydrochloric acid (1.5 M) to pH 4-5 and extracted with chloroform (4×30 mL). The combined organic layers were washed with water and brine, dried over sodium sulphate, filtered and concentrated in vacuo. The material was stirred with aqueous potassium hydroxide (1 M) for 4 hours and then acidified with aqueous hydrochloric acid (1.5 M) to pH 4-5, followed by extraction with ethyl-acetate (3×25 mL). The combined organic phases were washed with brine, dried over sodium sulphate, filtered and concentrated in vacuo to give 9-methyl-2-pyridin-2-yl-9H-purin-6-ol (750 mg, 21%) as the crude product.

### Example 12

### 6-Chloro-9-methyl-2-pyridin-2-yl-9H-purine (Intermediate compound)

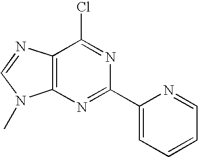

N,N-Diisopropylethylamine (1.26 mL, 7.26 mmol) and phosphorus oxychloride (0.62 mL, 6.60 mmol) were added dropwise to a solution of 9-methyl-2-pyridin-2-yl-9H-purin-6-ol (750 mg, 3.30 mmol) in acetonitrile (5 mL) and the mixture was heated at 70° C. for 3 days. The reaction mixture was basified with aqueous sodium hydrogen carbonate (10%) and extracted with ethyl acetate (4×20 mL). The combined organic layers were washed with brine, dried over sodium sulphate, filtered and concentrated in vacuo to give 6-chloro-9-methyl-2-pyridin-2-yl-9H-purine (650 mg, 80%) of the crude product.

### Example 13

### (4-Chloro-phenyl)-(9-methyl-2-pyridin-2-yl-9H-purin-6-yl)-amine (Compound 13.1)

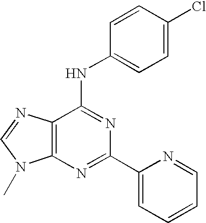

A solution of 6-chloro-9-methyl-2-pyridin-2-yl-9H-purine (600 mg, 2.44 mmol), N,N-diisopropylethylamine (0.64 mL, 3.37 mmol) and 4-chloroaniline (312 mg, 2.44 mmol) in acetonitrile (5 mL) were heated to 70° C. over night. The mixture was concentrated in vacuo and purified by column chromatography (chloroform/methanol) to give (4-chloro-phenyl)-(9-methyl-2-pyridin-2-yl-9H-purin-6-yl)-amine (150 mg, 20%) as a white solid. Mp. 185.4-190.5° C.

LC-ESI-HRMS of [M+H]+ shows 337.0959 Da. Calc. 337.096847 Da, dev. −2.8 ppm.

### Example 14

### Cyclohexyl-(9-methyl-2-pyridin-2-yl-9H-purin-6-yl)-amine (Compound 14.1)

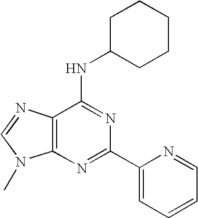

A solution of 6-chloro-9-methyl-2-pyridin-2-yl-9H-purine (300 mg, 1.22 mmol), N,N-diisopropylethylamine (0.32 mL, 1.83 mmol) and cyclohexylamine (0.15 mL, 1.34 mmol) in acetonitrile (5 mL) were stirred for three hours at room temperature. The mixture was concentrated in vacuo and purified by column chromatography (chloroform/methanol) to give cyclohexyl-(9-methyl-2-pyridin-2-yl-9H-purin-6-yl)-amine (215 mg, 57%) as a yellow solid.

LC-ESI-HRMS of [M+H]+ shows 309.1837 Da. Calc. 309.182769 Da, dev. 3 ppm.

### Example 15

### Biological Activity

The example below demonstrates the biological activity of the compounds of the invention. The ionic current through small-conductance Ca²⁺-activated K⁺ channels (SK channels, subtype 3) is recorded using the whole-cell configuration of the patch-clamp technique in a classic patch-clamp set-up using HEK293 tissue culture cells expressing hSK3 channels as described in e.g. WO 2006/100212.

The SC₁₀₀ value determined is defined as the Stimulating Concentration required for increasing the baseline current by 100%. The below SC₁₀₀ values are an indication of the SK3 activating properties of the compounds of the invention.

| Compound | The SC₁₀₀ (μM) |
|---|---|
| 6.1 | 0.3 |
| 6.2 | 0.18 |
| 6.3 | 0.7 |
| 6.6 | 0.02 |
| 7.1 | 0.6 |
| 9.1 | 0.02 |
| 9.3 | 0.6 |
| 9.5 | 0.15 |

1. A purinyl derivative of Formula Ia or Ib 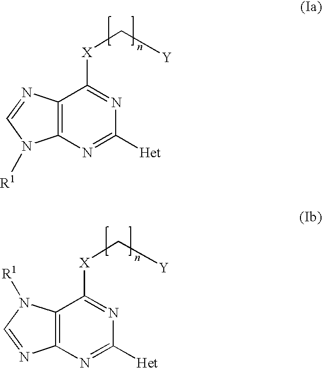
a stereoisomer thereof or a mixture of its stereoisomers, an N-oxide thereof, a prodrug thereof, or a pharmaceutically acceptable salt thereof, wherein
n is 0, 1, 2 or 3;
X represents O, S or NR′; wherein R′ represents hydrogen, alkyl, cycloalkyl, phenyl or benzyl;
Y represents alkyl, cycloalkyl or phenyl; which alkyl, cycloalkyl and phenyl are optionally substituted with one substituent selected from the group consisting of alkyl, cycloalkyl, halo, trifluoromethyl, trifluoromethoxy, hydroxy, alkoxy, cyano, nitro and amino;
R¹ represents hydrogen, alkyl or alkoxy-alkyl; and
Het represents a heterocyclic group selected from pyrazolyl, imidazolyl, indazolyl, benzimidazolyl, pyridinyl and cyclopentapyrazolyl, which pyrazolyl, imidazolyl indazolyl, benzimidazolyl, pyridinyl and cyclopentapyrazolyl may optionally be substituted with one substituent selected from the group consisting of alkyl, hydroxy-alkyl, cycloalkyl, cycloalkyl-alkyl, alkenyl, alkynyl, halo, trifluoromethyl, trifluoromethoxy, hydroxy, alkoxy, alkoxy-carbonyl, carboxy, cyano, nitro, amino, amino-carbonyl, N,N-dialkyl-amino-carbonyl, phenyl and benzyl.

2. The purinyl derivative of claim 1, a stereoisomer thereof or a mixture of its stereoisomers, an N-oxide thereof, a prodrug thereof, or a pharmaceutically acceptable salt thereof, wherein n is 0, 1 or 2.

3. The purinyl derivative claim 1, a stereoisomer thereof or a mixture of its stereoisomers, an N-oxide thereof, a prodrug thereof, or a pharmaceutically acceptable salt thereof, wherein X represents O, S or NR′; wherein R′ represents hydrogen or alkyl.

4. The purinyl derivative of claim 1, a stereoisomer thereof or a mixture of its stereoisomers, an N-oxide thereof, a prodrug thereof, or a pharmaceutically acceptable salt thereof, wherein Y represents cycloalkyl or phenyl; which cycloalkyl and phenyl are optionally substituted with one substituent selected from the group consisting of alkyl, cycloalkyl, halo, trifluoromethyl, trifluoromethoxy, hydroxy, alkoxy, cyano, nitro and amino.

5. The purinyl derivative of claim 1, a stereoisomer thereof or a mixture of its stereoisomers, an N-oxide thereof, a prodrug thereof, or a pharmaceutically acceptable salt thereof, wherein R¹ represents hydrogen or alkyl.

6. The purinyl derivative of claim 1, a stereoisomer thereof or a mixture of its stereoisomers, an N-oxide thereof, a prodrug thereof, or a pharmaceutically acceptable salt thereof, wherein Het represents a heterocyclic group selected from pyrazolyl, imidazolyl, indazolyl, benzimidazolyl, pyridinyl and cyclopentapyrazolyl, which pyrazolyl, imidazolyl, indazolyl, benzimidazolyl, pyridinyl and cyclopentapyrazolyl may optionally be substituted with one substituent selected from the group consisting of alkyl, hydroxy-alkyl, halo, trifluoromethyl, trifluoromethoxy, hydroxy, alkoxy, alkoxy-carbonyl, cyano, nitro, amino, phenyl and benzyl.

7. The purinyl derivative according to claim 6, a stereoisomer thereof or a mixture of its stereoisomers, an N-oxide thereof, a prodrug thereof, or a pharmaceutically acceptable salt thereof, wherein Het represents pyrazolyl, pyridinyl and cyclopentapyrazolyl, which pyrazolyl, pyridinyl and cyclopentapyrazolyl may optionally be substituted with one substituent selected from the group consisting of alkyl, hydroxy-alkyl, halo, trifluoromethyl, trifluoromethoxy, hydroxy, alkoxy, alkoxy-carbonyl, cyano, nitro, amino, phenyl and benzyl.

8. The purinyl derivative of claim 1, which is
(4-Chloro-phenyl)-[9-methyl-2-(3-trifluoromethyl-pyrazol-1-yl)-9H-purin-6-yl]-amine;
(4-Chloro-phenyl)-(9-methyl-2-pyrazol-1-yl-9H-purin-6-yl)-amine;
Cyclohexyl-(9-methyl-2-pyrazol-1-yl-9H-purin-6-yl)-amine;
(4-Chloro-phenyl)-[2-(4-chloro-pyrazol-1-yl)-9-methyl-9H-purin-6-yl]-amine;
(4-Chloro-phenyl)-[9-methyl-2-(4-methyl-pyrazol-1-yl)-9H-purin-6-yl]-amine;
(4-Fluoro-phenyl)-[9-methyl-2-(3-methyl-pyrazol-1-yl)-9H-purin-6-yl]-amine;
(4-Chloro-phenyl)-[9-methyl-2-(3-nitro-pyrazol-1-yl)-9H-purin-6-yl]-amine;
[2-(3-Amino-pyrazol-1-yl)-9-methyl-9H-purin-6-yl]-(4-chloro-phenyl)-amine;
(4-Chloro-phenyl)-[9-methyl-2-(3-methyl-pyrazol-1-yl)-9H-purin-6-yl]-amine;
[9-Methyl-2-(3-methyl-pyrazol-1-yl)-9H-purin-6-yl]-phenyl-amine;
[9-Methyl-2-(3-methyl-pyrazol-1-yl)-9H-purin-6-yl]-phenethyl-amine;
6-(4-Chloro-phenoxy)-9-methyl-2-(3-methyl-pyrazol-1-yl)-9H-purine;
(4-Fluoro-phenyl)-[9-methyl-2-(3-methyl-5,6-dihydro-4H-cyclopentapyrazol-2-yl)-9H-purin-6-yl]-amine;
(4-Chloro-phenyl)-(9-methyl-2-pyridin-2-yl-9H-purin-6-yl)-amine;
Cyclohexyl-(9-methyl-2-pyridin-2-yl-9H-purin-6-yl)-amine; or
a stereoisomer thereof or a mixture of its stereoisomers, an N-oxide thereof, a prodrug thereof, or a pharmaceutically acceptable salt thereof.

9. A pharmaceutical composition comprising a therapeutically-effective amount of a purinyl derivative according to claim 1, a stereoisomer thereof or a mixture of its stereoisomers, an N-oxide thereof, or a pharmaceutically-acceptable addition salt thereof, or a prodrug thereof, together with at least one pharmaceutically-acceptable carrier or diluent.

10. A method of treatment, prevention or alleviation of a disease or a disorder or a condition of a living animal body, including a human, which disease, disorder or condition is responsive to modulation of the potassium channels, and which method comprises:
administering to such a living animal body, including a human, in need thereof a therapeutically-effective amount of the purinyl derivative of claim 1, a stereoisomer thereof or a mixture of its stereoisomers, an N-oxide thereof, a prodrug thereof, or a pharmaceutically acceptable salt thereof.

11. The method according to claim 10, wherein the disease or a disorder associated with the activity of potassium channels is a respiratory disease, epilepsy, convulsions, seizures, absence seizures, vascular spasms, coronary artery spasms, renal disorders, polycystic kidney disease, bladder spasms, overactive bladder, urinary incontinence, bladder outflow obstruction, interstitiel cystitis, erectile dysfunction, gastrointestinal dysfunction, secretory diarrhoea, ischaemia, cerebral ischaemia, ischaemic heart disease, angina pectoris, coronary heart disease, autism, ataxia, traumatic brain injury, Parkinson's disease, bipolar disorder, psychosis, schizophrenia, anxiety, depression, mania, mood disorders, dementia, memory and attention deficits, Alzheimer's disease, amyotrophic lateral sclerosis (ALS), dysmenorrhea, narcolepsy, Reynaud's disease, intermittent claudication, Sjogren's syndrome, arrhythmia, hypertension, myotonic muscle dystrophia, spasticity, xerostomi, diabetes type II, hyperinsulinemia, premature labour, baldness, cancer, irritable bowel syndrome, immune suppression, migraine or pain, or withdrawal symptoms caused by the termination of abuse of chemical substances, in particular opioids, heroin, cocaine and morphine, benzodiazepines and benzodiazepine-like drugs, and alcohol.

12. (canceled)
